(19) United States
(12) Patent Application Publication
(10) Pub. No.: US 2021/0038085 A1
(43) Pub. Date: Feb. 11, 2021

(54) METHOD AND ELECTRONIC DEVICE CAPABLE OF MORE ACCURATELY ESTABLISHING PERSONAL BLOOD PRESSURE ESTIMATION MODEL FOR SPECIFIC USER BASED ON PERSONAL PROFILE OF PHYSIOLOGICAL FEATURE OF USER

(71) Applicant: PixArt Imaging Inc., Hsin-Chu City (TW)

(72) Inventor: Yuan-Hsin Liao, Hsin-Chu City (TW)

(21) Appl. No.: 17/080,862

(22) Filed: Oct. 27, 2020

Related U.S. Application Data
(63) Continuation-in-part of application No. 15/592,207, filed on May 11, 2017, now Pat. No. 10,849,565.

(51) Int. Cl. A61B 5/022; A61B 5/024; A61B 5/00

(52) U.S. Cl. CPC A61B 5/022; A61B 5/7246; A61B 5/02416; A61B 5/02438

(57) ABSTRACT

A method for establishing a personal blood pressure estimation model dedicated for a specific user includes: receiving a first reference measurement result of a reference sphygmomanometer; using a physiological sensor to measure the specific user's blood pressure to generate a first photoplethysmogram signal; calculating a first estimation result of the blood pressure of the specific user according to the first photoplethysmogram signal; generating a first regulating parameter by comparing the first reference measurement result with the first estimation result; and establishing the personal blood pressure estimation model by using the first regulating parameter to adjust a set of parameter factor(s) of a basic blood pressure estimation model.

### CROSS REFERENCE TO RELATED APPLICATIONS

This application is a continuation in part application and claims the benefit of U.S. Non-provisional application Ser. No. 15/592,207, which was filed on May 11, 2017 and is included herein by reference.

### BACKGROUND OF THE INVENTION

### 1. Field of the Invention

The invention relates to a physiological sensing mechanism, and more particularly to method and physiological sensor device for obtaining an estimation of a physiological feature of a specific user, method and physiological sensor device for establishing a profile of a physiological feature of a specific user, and method and system for establishing/managing/grading profiles corresponding to a physiological feature of different users.

### 2. Description of the Prior Art

Generally speaking, a conventional sensing device may estimate a user's physiological/biological feature such as blood pressure by referring to a physiological signal such as a photoplethysmogram signal. In order to obtain a precise estimation result, it is necessary for the waveform feature of the physiological signal to be clear and stable. However, actually, the waveform of the physiological signal is easily affected by other factors such as an external pressure exerted by a user's finger on a sensing area of conventional sensing device. For example, the conventional sensing device may be configured on a variety of smart phones, and different users can press/touch the sensing area to detect the blood pressure. Since the weights of the variety of smart phones are different and the different users have different operating behaviors or habits, each time the actual pressure exerted on the sensing area of conventional sensor device may be different. Through multiple experiments, the waveform features of physiological signals change and become different in response to externally exerted pressures, even more serious is that the waveform feature of a physiological signal may become unstable and disappear if the externally exerted pressure is too light or too heavy. Further, even the conventional sensor device may be used with a pressure sensor to detect the externally exerted pressure, it is also difficult to obtain a precise estimation result of the physiological feature since different users have different responses and portable device such as smart phones produced by different manufacturers may have different sizes and weights. Also, it is difficult to limit their operating behavior for users. Thus, it is difficult for the conventional sensor device to obtain precise estimation result(s) for a user or for different users.

Further, the estimation result of a specific user's blood pressure may be affected by the pressure asserted by the specific user on a physiological sensor.

### SUMMARY OF THE INVENTION

Therefore one of the objectives of the invention is to provide method and physiological sensor device for obtaining an estimation of a physiological feature of a specific user, method and physiological sensor device for establishing a profile of a physiological feature of a specific user, and method and system for establishing profiles corresponding to a physiological feature of different users, to solve the above-mentioned problems.

According to embodiments of the invention, a method for obtaining an estimation of a physiological feature of a specific user is disclosed. The method comprises: using a physiological sensor device under a normal sensing mode to sense the physiological feature of the specific user to generate a physiological signal of the normal sensing mode; determining a matched physiological signal from a plurality of test physiological signals by comparing the plurality of test physiological signals with the physiological signal of the normal sensing mode; and, calculating a resultant estimation of the physiological feature of the specific user according to the matched physiological signal and the profile of the physiological feature of the specific user.

According to the embodiments, a method for establishing a profile of a physiological feature of a specific user is further disclosed. The method comprises: using a physiological sensor device under a test sensing mode to sense the physiological feature of the specific user to obtain a plurality of observations for the physiological feature to generate a plurality of test physiological signals respectively; selecting an optimal test physiological signal among the plurality of test physiological signals; calculating a plurality of test estimations of the physiological feature of the specific user according to the plurality of test physiological signals; calculating a plurality of compensation parameters corresponding to the plurality of test physiological signals excluding the optimal test physiological signal by comparing a test estimation of the optimal test physiological signal with a test estimation of each different test physiological signal; and, establishing and storing the profile of the physiological feature of the specific user in the memory circuit according to the plurality of test physiological signals and the plurality of compensation parameters corresponding to the plurality of test physiological signals excluding the optimal test physiological signal.

According to the embodiments, a method for establishing profiles corresponding to a physiological feature of different users is disclosed. The method comprises: using a first physiological sensor device under a test sensing mode to sense the physiological feature of a first user to obtain a plurality of observations for the physiological feature of the first user to generate a plurality of first test physiological signals respectively; establishing a first profile corresponding to the physiological feature of the first user according to the plurality of first test physiological signals; using a second physiological sensor device under the test sensing mode to sense the physiological feature of a second user to obtain a plurality of observations for the physiological feature of the second user to generate a plurality of second test physiological signals respectively; establishing a second profile corresponding to the physiological feature of the second user according to the plurality of second test physiological signals; transmitting the first profile and the second profile to a remote system; and, assigning different grades to the physiological feature of the first user and the physiological feature of the second user according to the first profile and the second profile respectively.

According to the embodiments, a physiological sensor device for obtaining an estimation of a physiological feature of a specific user is disclosed. The physiological sensor device comprises a physiological sensor and a processing circuit. The physiological sensor is configured for operating under a normal sensing mode to sense the physiological feature of the specific user to generate a physiological signal of the normal sensing mode. The processing circuit is coupled to the physiological sensor and is configured for: determining a matched physiological signal from a plurality of test physiological signals by comparing the plurality of test physiological signals with the physiological signal of the normal sensing mode; and, calculating a resultant estimation of the physiological feature of the specific user according to the matched physiological signal and the profile of the physiological feature of the specific user.

According to the embodiments, a physiological sensor device for establishing a profile of a physiological feature of a specific user is disclosed. The physiological sensor device comprises a physiological sensor and a processing circuit. The physiological sensor is configured to operate under a test sensing mode to sense the physiological feature of the specific user to obtain a plurality of observations for the physiological feature to generate a plurality of test physiological signals respectively. The processing circuit is coupled to the physiological sensor and configured for: selecting an optimal test physiological signal among the plurality of test physiological signals; calculating a plurality of test estimations of the physiological feature of the specific user according to the plurality of test physiological signals; calculating a plurality of compensation parameters corresponding to the plurality of test physiological signals excluding the optimal test physiological signal by comparing a test estimation of the optimal test physiological signal with a test estimation of each different test physiological signal; and, establishing and storing the profile of the physiological feature of the specific user in the memory circuit according to the plurality of test physiological signals and the plurality of compensation parameters corresponding to the plurality of test physiological signals excluding the optimal test physiological signal.

According to the embodiments, a system for establishing profiles corresponding to a physiological feature of different users is disclosed. The system comprises a first physiological sensor device, a second physiological sensor device, and a processor. The first physiological sensor device is used for operating under a test sensing mode to sense the physiological feature of a first user to obtain a plurality of observations for the physiological feature of the first user to generate a plurality of first test physiological signals respectively. The second physiological sensor device is used for operating under the test sensing mode to sense the physiological feature of a second user to obtain a plurality of observations for the physiological feature of the second user to generate a plurality of second test physiological signals respectively. The processor is used for establishing a first profile corresponding to the physiological feature of the first user according to the plurality of first test physiological signals, establishing a second profile corresponding to the physiological feature of the second user according to the plurality of second test physiological signals, and assigning different grades to the physiological feature of the first user and the physiological feature of the second user according to the first profile and the second profile respectively.

According to the embodiments of the invention, a method to be executed on a mobile device, a handheld device, or a wearable electronic device and arranged for establishing a personal blood pressure estimation model dedicated for a specific user operating the mobile device, the handheld device, or the wearable electronic device, is disclosed. The method comprises: receiving a first reference measurement result generated by a reference sphygmomanometer which is a larger external electronic device being not like the mobile device, the handheld device, and the wearable electronic device; using a physiological sensor, installed on the mobile device, the handheld device, or the wearable electronic device, to measure the specific user's blood pressure to generate a first photoplethysmogram signal to calculate a first estimation result of the specific user's blood pressure, comprising: using the physiological sensor under a normal sensing mode to sense the specific user's blood pressure to generate the first photoplethysmogram signal of the normal sensing mode; determining a matched photoplethysmogram signal from a plurality of test photoplethysmogram signals by comparing the plurality of test photoplethysmogram signals with the first photoplethysmogram signal of the normal sensing mode; when the matched photoplethysmogram signal is an optimal test photoplethysmogram, calculating the first estimation result of the specific user's blood pressure according to the matched photoplethysmogram signal; and when the matched photoplethysmogram signal is a non-optimal test photoplethysmogram signal in the plurality of test photoplethysmogram signals and different from the optimal test photoplethysmogram signal, calculating a preliminary estimation of the specific user's blood pressure according to the non-optimal test photoplethysmogram signal and then compensating the preliminary estimation by referring to a compensation parameter corresponding to the non-optimal test photoplethysmogram signal without using the optimal test photoplethysmogram signal so as to generate the first estimation result of the specific user's blood pressure, wherein a plurality of non-optimal test physiological signals in the plurality of test photoplethysmogram signals are respectively generated in response to different pressures asserted by the specific user on the physiological sensor; and generating a first regulating parameter by comparing the first reference measurement result with the first estimation result to calculate a first difference between the first reference measurement result and the first estimation result as the first regulating parameter; establishing a set of parameter factor(s) of the personal blood pressure estimation model dedicated for the specific user operating the mobile device, the handheld device, or the wearable electronic device by using the first regulating parameter to adjust a set of parameter factor(s) of a basic blood pressure estimation model implemented on the mobile device wherein the set of parameter factor(s) of the basic blood pressure estimation model are used and identical for the specific user and another different user while the set of parameter factor(s) of the personal blood pressure estimation model is merely used for the specific user and is not used for the another different user; and using the physiological sensor with the set of parameter factor(s) of the established personal blood pressure estimation model dedicated for the specific user to measure the specific user's blood pressure to generate blood pressure information without asking the specific user to operate the reference sphygmomanometer.

According to the embodiments, an electronic device being a mobile device, a handheld device, or a wearable electronic device and arranged for establishing a personal blood pressure estimation model dedicated for a specific user operating the mobile device, the handheld device, or the wearable electronic device is disclosed. The electronic device comprises a receiving unit and a physiological sensor device. The physiological sensor device comprises a physiological sensor and a processing circuit. The receiving unit is configured to receive a first reference measurement result of a reference sphygmomanometer which is a larger external electronic device being not like the mobile device, the handheld device, and the wearable electronic device. The physiological sensor is coupled to the receiving unit and installed on the mobile device, the handheld device, or the wearable electronic device, and it is configured to measure the specific user's blood pressure to generate a first photoplethysmogram signal. The processing circuit is coupled to the receiving unit and the physiological sensor, and it is configured to: calculate a first estimation result of the first blood pressure of the specific user according to the first photoplethysmogram signal; generate a first regulating parameter by comparing the first reference measurement result with the first estimation result to calculate a first difference between the first reference measurement result and the first estimation result as the first regulating parameter; establish a set of parameter factor(s) of the personal blood pressure estimation model dedicated for the specific user operating the mobile device, the handheld device, or the wearable electronic device by using the first regulating parameter to adjust a set of parameter factor(s) of a basic blood pressure estimation model implemented on the mobile device wherein the set of parameter factor(s) of the basic blood pressure estimation model are used and identical for the specific user and another different user while the set of parameter factor(s) of the personal blood pressure estimation model is merely used for the specific user and is not used for the another different user; and use the physiological sensor device with the established personal blood pressure estimation model dedicated for the specific user to measure the specific user's blood pressure to generate blood pressure information without asking the specific user to operate the reference sphygmomanometer. The physiological sensor is arranged for sensing the specific user's blood pressure under a normal sensing mode to generate the first photoplethysmogram signal of the normal sensing mode; and, the processing circuit is arranged for determining a matched photoplethysmogram signal from a plurality of test photoplethysmogram signals by comparing the plurality of test photoplethysmogram signals with the first photoplethysmogram signal of the normal sensing mode; when the matched photoplethysmogram signal is an optimal test photoplethysmogram signal, the processing circuit calculates the first estimation result of the specific user's blood pressure according to the matched photoplethysmogram signal; and, when the matched photoplethysmogram signal is a non-optimal test photoplethysmogram signal in the plurality of test photoplethysmogram signals and different from the optimal test photoplethysmogram signal, the processing circuit calculates a preliminary estimation of the specific user's blood pressure according to the non-optimal test photoplethysmogram signal and then compensates the preliminary estimation by referring to a compensation parameter corresponding to the non-optimal test photoplethysmogram signal without using the optimal test photoplethysmogram signal so as to generate the first estimation result of the specific user's blood pressure, wherein a plurality of non-optimal test physiological signals in the plurality of test photoplethysmogram signals are respectively generated in response to different pressures asserted by the specific user on the physiological sensor.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram illustrating a flowchart for obtaining an estimation of a physiological feature of a specific user under a normal sensing mode according to a first embodiment of the invention.

FIG. 2 is a diagram of a physiological sensor device for obtaining an estimation of a physiological feature of a specific user according to the embodiment as shown in FIG. 1.

FIG. 3 is a diagram illustrating a flowchart for generating or establishing information of a profile of a specific user under the test sensing mode according to a second embodiment of the invention.

FIG. 4 is a diagram illustrating examples of two different users pressing a touch sensing area of physiological sensor several times by putting different pressures on the touch sensing area.

FIG. 5 is a diagram illustrating a flowchart of an example procedure for establishing/managing profiles corresponding to physiological feature(s) of different users according to a third embodiment of the invention.

FIG. 6 is a diagram of a system for establishing/managing profiles corresponding to physiological feature(s) of different users according to the embodiment of FIG. 5.

FIG. 7 is a diagram showing a flowchart of a method used for establishing or generating a personal blood pressure estimation model of a specific user/person according to embodiments of the invention.

FIG. 8 is a diagram illustrating an electronic device used for establishing or generating the personal blood pressure estimation model of the specific user/person according to the embodiment flowchart of FIG. 7.

FIG. 9 is a diagram showing an example of one PPG waveform signal.

### DETAILED DESCRIPTION

The mechanism provided by embodiments of the invention is arranged to more accurately obtain an estimation of physiological feature of a specific user by performing only one observation (i.e. one-time observation) for the physiological feature of the specific user wherein the physiological feature for example comprises the user's blood pressure, heart rate, and/or other physiological/biological characteristics. This mechanism is especially useful for obtaining an accurate estimation of the physiological feature under a condition that the physiological feature is not easily detected or estimated due to different user's behaviors. For instance, a conventional physiological sensor device may be used for detecting a user's blood pressure, and the user can touch or press a touch sensing area of the conventional physiological sensor device to sense the user's blood pressure. However, actually, the conventional physiological sensor device needs to perform many observations for the user's blood pressure since the detection result is easily affected by different behaviors of the user (i.e. the user may press or touch the sensing area with different pressures each time). In addition, different users also include different pressing behaviors with different pressures, and the same conventional physiological sensor device may not be suitable for different users. It is difficult to accurately estimate a user's blood pressure by only one observation of the conventional physiological sensor device. The methods and physiological sensor devices provided by the embodiments are to solve the above-mentioned problems. Description is detailed in the following.

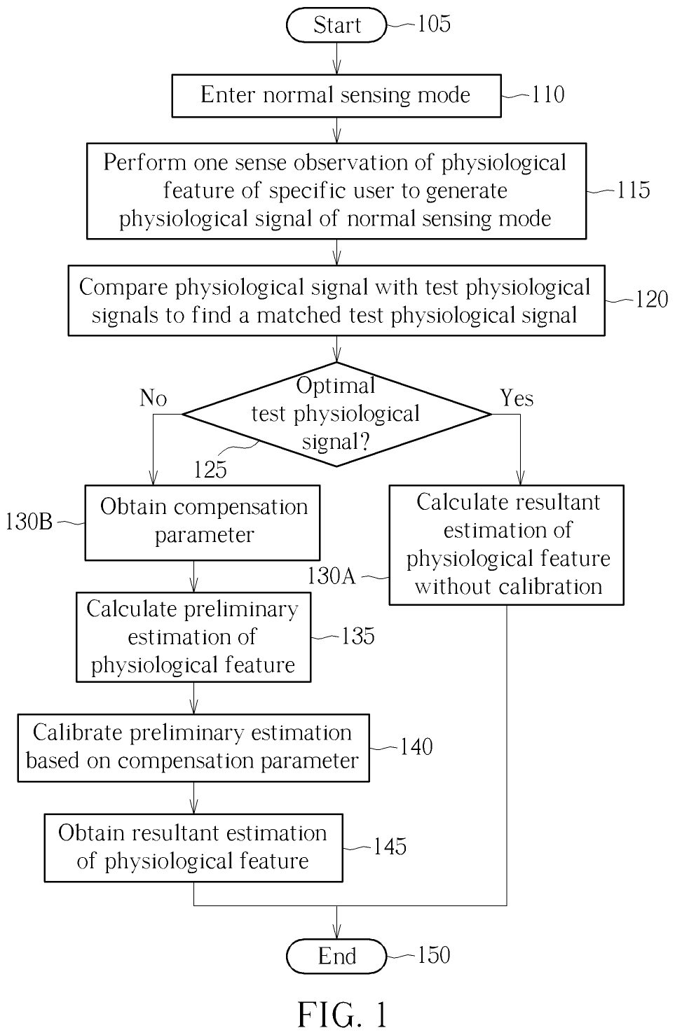

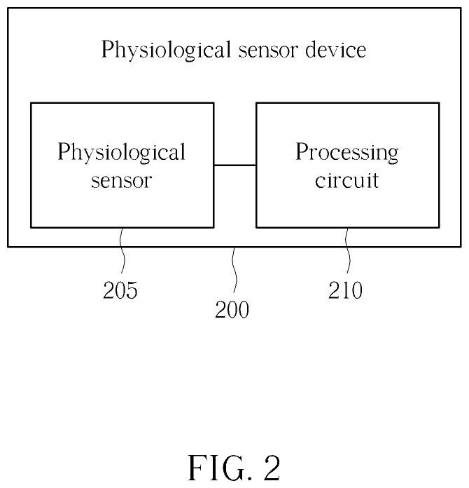

Refer to FIG. 1 in conjunction with FIG. 2. FIG. 1 is a diagram illustrating a flowchart for obtaining an estimation of a physiological feature of a specific user under a normal sensing mode according to a first embodiment of the invention. FIG. 2 is a diagram of a physiological sensor device 200 for obtaining an estimation of a physiological feature of a specific user according to the embodiment as shown in FIG. 1. The physiological sensor device 200 comprises a physiological sensor 205 and a processing circuit 210 such as a microprocessor, microcontroller, or a processor. The physiological feature of the specific user, for example, indicates a blood pressure of the specific user (but not limited); in other examples, the physiological feature may indicate the heart rate of the user. Provided that substantially the same result is achieved, the steps of the flowchart shown in FIG. 1 need not be in the exact order shown and need not be contiguous, that is, other steps can be intermediate. Steps are detailed in the following:

Step 105: Start;

Step 110: The physiological sensor device 200 enter and operate under the normal sensing mode;

Step 115: Use the physiological sensor device 200 operating under the normal sensing mode to perform one sense observation of the physiological feature of the specific user to generate/obtain a physiological signal of the specific user under the normal sensing mode;

Step 120: Compare the physiological signal of the specific user with a plurality of test physiological signals of the specific user by using the processing circuit 210 to find a matched test physiological signal from the plurality of test physiological signals;

Step 125: Determine whether the matched test physiological signal is an optimal test physiological signal among the test physiological signals. If the matched test physiological signal is an optimal test physiological signal, the flow proceeds to Step 130A, otherwise the flow proceeds to Step 130B;

Step 130A: Calculate a resultant estimation of the physiological feature of the specific user by using the processing circuit 210 according to the matched physiological signal without calibration;

Step 130B: obtain or retrieve a compensation parameter associated with the matched test physiological signal which is not an optimal test physiological signal;

Step 135: Calculate a preliminary estimation of the physiological feature of the specific user by using the processing circuit 210 according to the matched physiological signal;

Step 140: Calibrate or compensate the preliminary estimation of the physiological feature of the specific user by using the processing circuit 210 based on the obtained/retrieved compensation parameter;

Step 145: Obtain or generate a resultant estimation of the physiological feature of the specific user;

Step 150: End.

As mentioned in Step 115, the physiological sensor 205 is configured for operating under the normal sensing mode to sense the physiological feature of the specific user to generate the physiological signal of the normal sensing mode. The processing circuit 210 is coupled to the physiological sensor 205, and is configured for determining the matched physiological signal from the test physiological signals by comparing the test physiological signals with the physiological signal of the normal sensing mode (Step 120), and for calculating a resultant estimation of the physiological feature of the specific user according to the matched physiological signal and a profile of the physiological feature of the specific user (Step 125-Step 145) wherein the profile of the specific user means test physiological signals, compensation parameter(s), and optimal test physiological signal which are associated with such user. Information of different users' profiles is different. Further, a physiological signal for example is a photoplethysmogram signal (but not limited).

The physiological sensor device 200 comprises the normal sensing mode and a test sensing mode. Under the normal sensing mode, the physiological sensor device 200 is used to generate the resultant estimation of the physiological feature for the specific user. Under the test sensing mode, the physiological sensor device 200 is arranged to generate/establish the above-mentioned profile for the specific user.

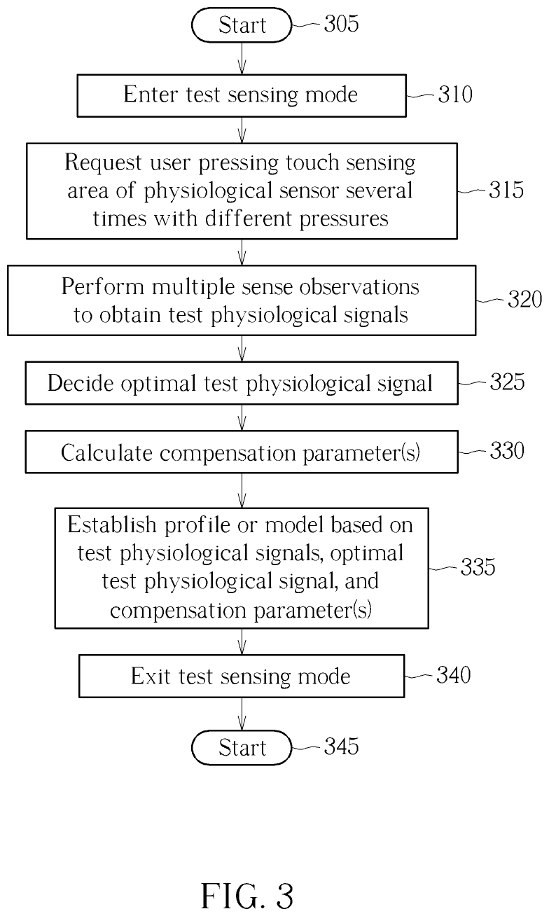

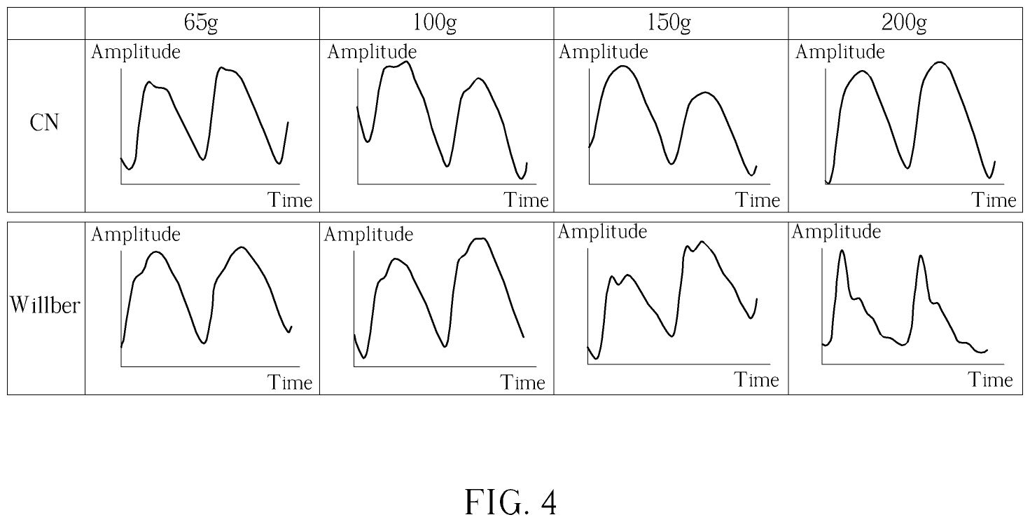

Refer to FIG. 3 in conjunction with FIG. 4. FIG. 3 is a diagram illustrating a flowchart for generating or establishing information of a profile of a specific user under the test sensing mode according to a second embodiment of the invention. FIG. 4 is a diagram illustrating examples of two different users pressing a touch sensing area of physiological sensor 205 several times by putting different pressures on the touch sensing area. Provided that substantially the same result is achieved, the steps of the flowchart shown in FIG. 3 need not be in the exact order shown and need not be contiguous, that is, other steps can be intermediate. Steps are detailed in the following:

Step 305: Start;

Step 310: The physiological sensor device 200 enter and operate under the test sensing mode;

Step 315: The physiological sensor device 200 via a human-machine interface asks or requests a specific user pressing the touch sensing area of physiological sensor 205 several times with different pressures on the touch sensing area;

Step 320: The physiological sensor 205 performs multiple sense observations to obtain and generate the test physiological signals;

Step 325: The processing circuit 210 decides an optimal test physiological signal from the test physiological signals;

Step 330: The processing circuit 210 calculates/derives compensation parameter(s) corresponding to the other test physiological signals, i.e. the test physiological signals excluding the optimal test physiological signal;

Step 335: The processing circuit 210 establishes and completes the profile or model based on the test physiological signals, optimal test physiological signal, and the compensation parameter(s);

Step 340: Exit the test sensing mode; and

Step 345: End.

Specifically, in Step 315 and Step 320, for example, the physiological sensor device 200 can be further designed with the human-machine interface which is capable of asking a user to touch/press the touch sensing area with different pressures, and the physiological sensor 205 senses the physiological feature of the user several times to obtain a plurality of observations for the physiological feature to generate the test physiological signals respectively. FIG. 4 shows examples of two different users CN and Willber respectively pressing the touch sensing area of physiological sensor 205 by putting different pressures on the touch sensing area. In FIG. 4, the vertical axis means the amplitude of waveform of each physiological signal, and the horizontal axis means the time index.

For instance, in Step 315, the human-machine interface can request a user (CN or Willber) to press the touch sensing area with different pressures four times. As shown by FIG. 4, if the specific user (a current user) is user CN, different patterns of four test physiological signals corresponding to four different pressures such as 65 grams, 100 grams, 150 grams, and 200 grams, respectively asserted/pressed by the users CN, are generated by the physiological sensor 205. Alternatively, if the current user is user Willber, different patterns of another four test physiological signals corresponding to four different pressures such as 65 grams, 100 grams, 150 grams, and 200 grams, respectively asserted/pressed by the users Willber, are generated by the physiological sensor 205. It should be noted that the above-mentioned pressures are not meant to be limitations. Further, it can be seen from different patterns of FIG. 4 that the physiological sensor device 200 can establish different profiles for different users such as users CN and Willber.

In Step 325, the processing circuit 210 selects an optimal test physiological signal among the plurality of test physiological signals. In practice, the processing circuit 210 can determine the optimal test physiological signal based on the waveforms or patterns of test physiological signals. A test physiological signal can be selected as the optimal signal if it is easier or more suitable to use such physiological signal compared to using the other signals to precisely estimate the physiological feature of a user. If more than two test physiological signals are suitable, then either of the test physiological signals can be selected as the optimal test signal. In practice, for example, the processing circuit 210 may select the optimal test physiological signal according to a slope change/reverse of values neighboring to a peak value of each test physiological signal. For instance, as shown in FIG. 4, for the case of the current user being user Willber, four test physiological signals including different patterns are generated wherein the slope of values neighboring to a peak value of a physiological signal corresponding to the pressure of 150 grams clearly changes or reverses from a negative value to a positive value and this may indicate a reflection wave for the blood pressure. Thus, in this example for estimating the physiological feature of blood pressure, the processing circuit 210 is arranged for selecting a physiological signal including a clear/severe slope change/reverse neighboring to or at its peak value as the optimal test physiological signal. However, this is not meant to be a limitation. In another example for estimating other physiological features, the processing circuit 210 can be arranged for selecting a physiological signal having a waveform cycle with more than a particular time period as the optimal test physiological signal.

After determining the optimal test physiological signal, in Step 330, the processing circuit 210 is arranged to calculate compensation parameters corresponding to the other test physiological signals, i.e. all test physiological signals excluding the optimal test physiological signal. For example, the processing circuit 210 generates and calculates three compensation parameters for the test physiological signals associated with the pressures of 65 grams, 100 grams, and 200 grams corresponding to the user Willber. The three compensation parameters are respectively used for calibration or compensation. In practice, the processing circuit 210 may be arranged to calculate a plurality of test estimations (i.e. test estimation results) of test physiological signals, and then generates compensation parameters based on the optimal test physiological signal and a simple linear statistics model/equation. Taking the example of the current user being user Willber, the processing circuit 210 selects the test physiological signal corresponding to the pressure of 150 grams as the optimal test signal, and then based on the selected optimal test signal and the simple linear statistics model/equation the processing circuit 210 for example generates a linear parameter as the compensation parameter of the pressure 200 grams by using the simple linear statistics model/equation to perform calculations upon the test estimations of test physiological signals of pressures 150 grams and 200 grams. Similarly, the processing circuit 210 can generate linear compensation parameters for the pressures 65 grams and 100 grams. It should be noted that the simple linear statistics model/equation may be represented by A×Δt+B=C wherein parameters A and B means fixed or predefined parameters while Δt means a time width of pulse wave of a test physiological signal and parameter C means a test estimation result of such test physiological signal. Parameters Δt and C are varied with different patterns of different test physiological signals. Based on this simple linear statistics model/equation, the processing circuit 210 can rapidly calculate the corresponding compensation parameters. However, the example of simple linear statistics model/equation is not meant to be a limitation; in other examples, the processing circuit 210 can adopt different algorithms/models/equations to derive the corresponding compensation parameters.

Further, the above-described profile can be established when a portable device carrying the physiological sensor device 200 is enabled/activated and/or after such portable device has been enabled.

Thus, after the profile of a specific user such as user Willber has been established or completed, the physiological sensor device 200 operating under the normal sensing mode can accurately estimate the physiological feature of the user Willber by performing only one sense observation for the user's physiological feature. For example, under the normal sensing mode, if the physiological sensor device 200 generates and obtains a physiological signal which is like to or matched to the optimal test physiological signal such as the test physiological signal corresponding to 150 grams pressed by the user Willber, the processing circuit 210 generate a resultant estimation based on such physiological signal without calibrating or compensating the resultant estimation. If the physiological signal which is like to or matched to the test physiological signal such as the signal corresponding to 200 grams, the processing circuit 210 generates a preliminary estimation based on the physiological signal and then uses the compensation parameter associated with the test physiological signal of the pressure 200 grams to compensate/calibrate the preliminary estimation to generate a resultant estimation. By doing so, when the physiological sensor device 200 operates under the normal sensing mode to detect the blood pressure of a user, no matter how the user presses/touches a touch sensing area of the sensor device 200, the sensor device 200 is capable of generating a precise estimation for the user's blood pressure. That is, under the normal sensing mode, the sensor device 200 can generate the precise/accuracy estimation result for the user's blood pressure by performing only one observation for the physiological feature. This obtains a great performance improvement since a conventional sensor device requires performing multiple observations for the physiological feature to obtain a precise/accuracy estimation result.

Further, the processing circuit 210 may store the profile of the physiological feature of the specific user in the memory circuit, which can be configured within the physiological sensor device 200 or configured outside the physiological sensor device 200. For example, the profile of the physiological feature of the specific user may be stored in a flash memory circuit or a remote system. Additionally, a profile of the physiological feature of the specific user can be stored in an external memory circuit or a remote system and can be downloaded into the physiological sensor device 200, so that the physiological sensor device 200 can obtain the profile information without entering the test sensing mode to generate such information.

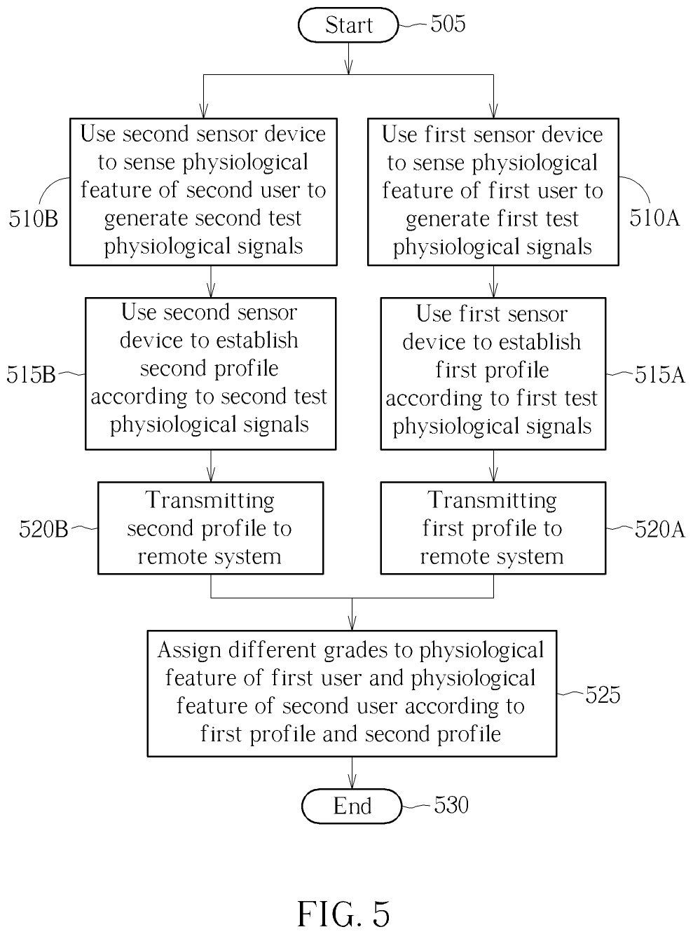

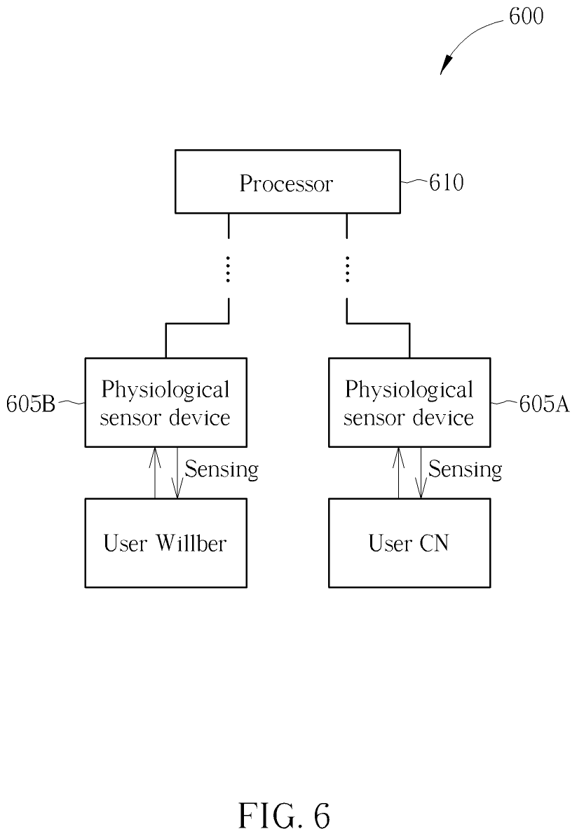

Additionally, in other embodiments, profiles of different users can be transmitted to and managed by a remote system which can assign different grades for different patterns and/or different profiles of the different users for big data analysis. Refer to FIG. 5 in conjunction with FIG. 6. FIG. 5 is a diagram illustrating a flowchart of an example procedure for establishing/managing profiles corresponding to physiological feature(s) of different users according to a third embodiment of the invention. FIG. 6 is a diagram of a system 600 for establishing/managing profiles corresponding to physiological feature(s) of different users according to the embodiment of FIG. 5. The system 600 comprises a processor 610 and at least two physiological sensor devices such as first physiological sensor device 605A and second physiological sensor device 605B which are externally wireless/wire connected to the processor 610. Provided that substantially the same result is achieved, the steps of the flowchart shown in FIG. 5 need not be in the exact order shown and need not be contiguous, that is, other steps can be intermediate. Steps are detailed in the following:

Step 505: Start;

Step 510A: Use the first physiological sensor device 605A under the test sensing mode to sense the physiological feature of a first user such as user CN to obtain a plurality of observations for the physiological feature of the user CN to generate a plurality of first test physiological signals respectively;

Step 510B: Use the second physiological sensor device 605B under the test sensing mode to sense the physiological feature of a second user such as user Willber to obtain a plurality of observations for the physiological feature of user Willber to generate a plurality of second test physiological signals respectively;

Step 515A: Use the first physiological sensor device 605A to establish a first profile corresponding to the physiological feature of the user CN according to the plurality of first test physiological signals;

Step 515B: Use the second physiological sensor device 605B to establish a second profile corresponding to the physiological feature of the user Willber according to the plurality of second test physiological signals;

Step 520A: transmitting the first profile to the processor 610 of remote system 600;

Step 520B: transmitting the second profile to the processor 610 of remote system 600;

Step 525: Use the processor 610 to assigning different grades to the physiological feature of the first user CN and the physiological feature of the second user Willber according to the first profile and the second profile respectively; and

Step 530: End.

The operations and functions of physiological sensor devices 605A and 605B are identical to that of physiological sensor device 200 and it not detailed for brevity. In addition, in another embodiment, the operations of Step 515A and Step 515B can be performed by the processor 610 of remote system 600. That is, the physiological sensor devices can directly transmit the information of test physiological signals to the remote processor 610 without generating the profiles, and calculations of the profiles are performed by the processor 610.

Additionally, the above-mentioned methods may be employed and applied into electronic devices to be capable of establishing personal physiological characteristics estimation models for different persons/users, i.e. to provide/generate different estimation models respectively dedicated for different persons. The methods can calibrate or adjust parameter factor(s) of a preliminary/preset/basic physiological characteristics estimation model (e.g. estimation curve) by referring to a user's physiological characteristics result(s) measured by a physiological sensor such as a photoplethysmogram (PPG) sensor and the user's physiological characteristics result(s) measured by a reference physiological characteristics detection device, to generate and provide a personal physiological characteristics estimation model dedicated to such user, after the profiles of the different persons are established. Thus, the physiological characteristics of the user can be precisely and accurately estimated by using his/her personal physiological characteristics estimation model of the electronic device belonging to the user.

For example, the physiological characteristics mean the user's blood pressure. The preliminary/preset/basic physiological characteristics estimation model means a preliminary/preset/basic blood pressure estimation model, and the personal physiological characteristics estimation model means a personal/dedicated blood pressure estimation model for a particular/specific user. The reference physiological characteristics detection device may be a reference sphygmomanometer (meter or monitor); however, this is not intended to be a limitation. The sphygmomanometer may be implemented using various kinds of sphygmomanometers which may be produced by different manufacturers such as Omron Healthcare Company or other manufacturers.

In the embodiments, the reference sphygmomanometer indicates a larger electronic device which can generate reference/accurate measurement result(s) for the blood pressure of the user; the reference sphygmomanometer is not like a mobile device, a handheld device or a wearable electronic device. The method is to provide the PPG sensor with the basic blood pressure estimation model, use the PPG sensor with the basic blood pressure estimation model to obtain estimation result(s) of the blood pressure of the user, and finally to fix/calibrate/adjust parameter factor(s) of such basic estimation model based on the reference/accurate measurement result(s) and the obtained estimation result(s) to generate the personal or dedicated blood pressure estimation model.

It should be noted that, to obtain precise/accurate parameter factor(s) of personal or dedicated blood pressure estimation model, the method may be arranged to ask the user to operate the reference sphygmomanometer and the PPG sensor based on identical/similar user behaviors so as to obtain more accurate/precise parameter factor(s) as far as possible. For instance, the method may be arranged to ask the user to operate the reference sphygmomanometer and the PPG sensor after he or she has took a rest. However, this is not meant to be a limitation.

In addition, the same type of device incorporating the above method may be operated by different users, even though the parameter factor(s) of the basic estimation model is/are identical, the method can be arranged to adjust the parameter factor(s) as different parameter factor(s) based on different users' physiological characteristics to generate different personal estimation models respectively dedicated to different persons.

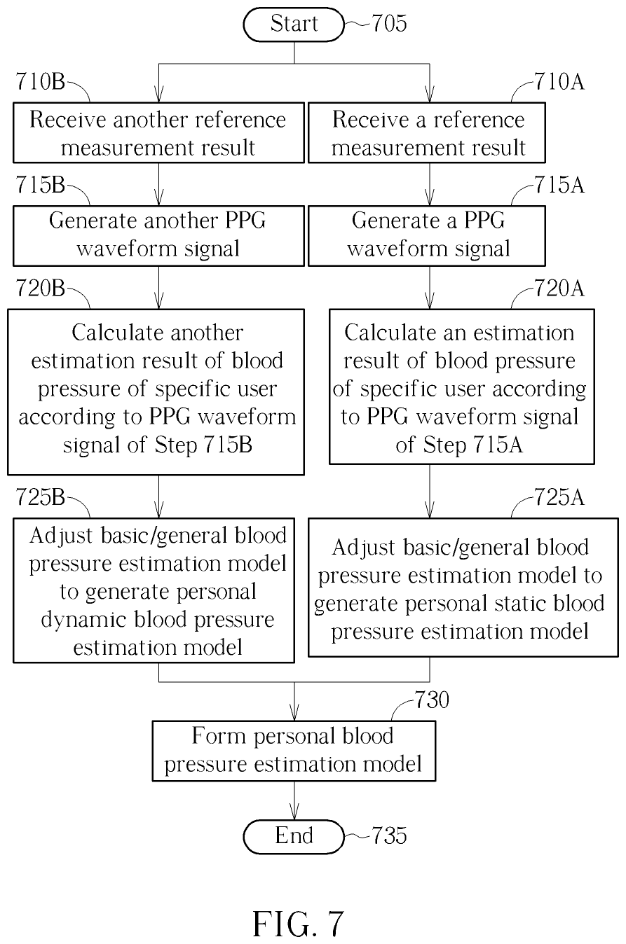

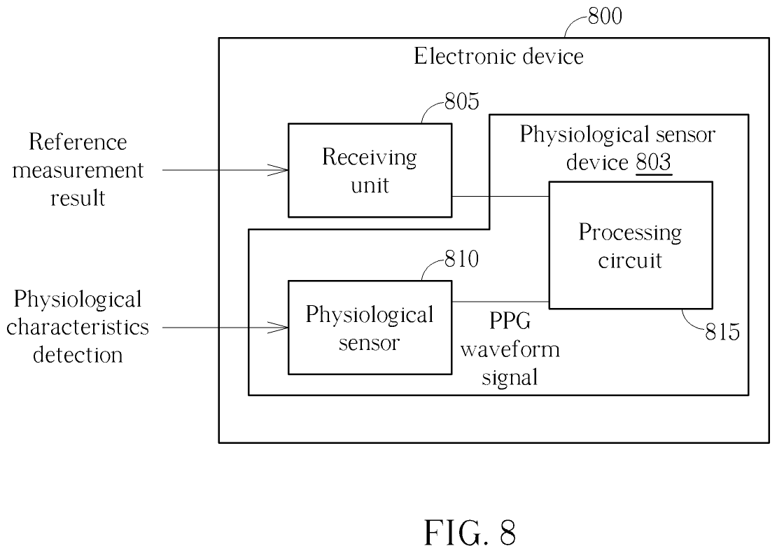

Refer to FIG. 7 in conjunction with FIG. 8. FIG. 7 is a diagram showing a flowchart of a method used for establishing or generating a personal blood pressure estimation model of a specific user/person according to embodiments of the invention. FIG. 8 is a diagram illustrating an electronic device 800 used for establishing or generating the personal blood pressure estimation model of the specific user/person according to the embodiment flowchart of FIG. 7. The electronic device 800 such as a mobile device, a handheld device, a wearable electronic device, or an interactive human-machine interface device, and comprises a physiological sensor device 803 and a receiving unit 805 (hardware circuit, software element, or combinations). The physiological sensor device 803 includes the operations and functions identical to those of physiological sensor device 200 and further includes the operation of establishing or generating a personal blood pressure estimation model of a specific user/person. In practice, the physiological sensor device 803 comprises a physiological sensor 810 such as a PPG sensor and a processing circuit 815 such as a processor; the reference sphygmomanometer is not shown on FIG. 8.

The electronic device 800 is capable of establishing or generating a personal blood pressure estimation model of a specific user based on a basic/general blood pressure estimation model and then using the personal blood pressure estimation model to measure/detect the specific user's blood pressure to obtain more accurate blood pressure information. Provided that substantially the same result is achieved, the steps of the flowchart shown in FIG. 7 need not be in the exact order shown and need not be contiguous, that is, other steps can be intermediate. Steps are detailed in the following:

Step 705: Start;

Step 710A: Use the receiving unit 805 to receive a reference measurement result of the reference sphygmomanometer wherein the reference measurement result is obtained when the change of blood pressure of specific user is at a linear state;

Step 710B: Use the receiving unit 805 to receive another reference measurement result of the reference sphygmomanometer wherein the another reference measurement result is obtained when the change of blood pressure of specific user is at a non-linear state;

Step 715A: Use the physiological sensor 810 to detect the physiological characteristics of the specific user for one time to generate a PPG waveform signal when the change of blood pressure of specific user is at the linear state;

Step 715B: Use the physiological sensor 810 to detect the physiological characteristics of the specific user for one time to generate a PPG waveform signal when the change of blood pressure of specific user is at the non-linear state;

Step 720A: Use the processing circuit 815 to calculate and obtain an estimation result of the blood pressure of the specific user according to the generated the PPG waveform signal of Step 715A;

Step 720B: Use the processing circuit 815 to calculate and obtain another estimation result of the blood pressure of the specific user according to the generated the PPG waveform signal of Step 715B;

Step 725A: Adjust a set of parameter factor(s) of the basic/general blood pressure estimation model used by the physiological sensor 810 by comparing the reference measurement result of Step 710A with the estimation result of blood pressure of Step 720A, to generate the personal static blood pressure estimation model dedicated for the specific user;

Step 725B: Adjust another set of parameter factor(s) of the basic/general blood pressure estimation model used by the physiological sensor 810 by comparing the reference measurement result of Step 710B with the estimation result of blood pressure of Step 720B, to generate the personal dynamic blood pressure estimation model dedicated for the specific user;

Step 730: Generate the personal blood pressure estimation model based on the personal static blood pressure estimation model and personal dynamic blood pressure estimation model; and

Step 735: End.

It should be noted that before or when the physiological sensor 810 in Step 715A or Step 715B is used to detect the physiological characteristics of the specific user, e.g. the specific user's blood pressure, to generate a PPG waveform signal, the physiological sensor device 803 can generate the above mentioned profile for the specific user based on the steps of the flowchart of FIG. 3. In addition, when generate one or more physiological signals such as PPG waveform signals, the physiological sensor device 803 can operate based on the steps of the flowchart of FIG. 1 to generate a resultant estimation result of the specific user's physiological feature such as blood pressure. Thus, even though the specific user may assert different pressures to or may touch the electronic device 800 with different pressures, the electronic device 800 can still generate an estimation result of the specific user's blood pressure, which is not affected by the asserted different touch/hand pressures. The personal blood pressure estimation model which is established or generated based on the more accurate estimation results can be more accurately generated and provided for the specific user. Further, in the embodiments, physiological signal(s) may mean photoplethysmogram signal(s), and similarly test, optimal, non-optimal physiological signal(s) respectively mean test, optimal, non-optimal photoplethysmogram signal(s).

In the embodiments, a reference measurement result has a reference value of systolic blood pressure and a reference value of diastolic blood pressure, and an estimation result of blood pressure has an estimation value of systolic blood pressure and an estimation value of diastolic blood pressure. That is, the electronic device 800 incorporating the method is arranged to estimate the specific user's systolic blood pressure and diastolic blood pressure. This is not intended to be a limitation. In another embodiment, the electronic device 800 may be arranged to estimate one of the systolic blood pressure and diastolic blood pressure.

Additionally, the change of blood pressure of a specific user may be at a steady state and at a non-steady state respectively corresponds to different behaviors/actions of the specific user. For example, the steady state may mean that the specific user takes a rest for a few minutes. The non-steady state has two different possible states comprising the above-mentioned linear state and non-linear state. The linear state means that the values of specific user's systolic and diastolic blood pressures are linearly changed with the heart rate of the specific user, and the non-linear state means that the values of specific user's systolic and diastolic blood pressures are non-linearly changed with the heart rate of the specific user.

For example, in a default setting of the electronic device 800, the linear state may mean that the specific user participates in daily activities such as brushing teeth and washing face (but not limited) and/or indoor activities (excluding exercise) such as playing cards or walking around a room slowly, and so on. The linear state is associated with the specific user's static blood pressure estimation. In the default setting, the non-linear state may mean that the specific user participates in indoor exercise activities (e.g. dancing, rock climbing, or others) and/or outdoor activities such as exercising, jogging, or catching a bus, and so on. The non-linear state is associated with the specific user's dynamic blood pressure estimation. Generating the personal blood pressure estimation model dedicated for the specific user comprises generating a personal static blood pressure estimation model and/or a personal dynamic blood pressure estimation model.

For generating the personal static blood pressure estimation model and the personal dynamic blood pressure estimation model, in Step 710A and Step 710B, the electronic device 800 is arranged to control the receiving unit 805 to at least receive two reference measurement results which are measured and obtained by the reference sphygmomanometer when the change of blood pressure of specific user is at the linear state and non-linear state respectively.

Correspondingly, in Step 715A and Step 715B, the electronic device 800 is arranged to control and use the physiological sensor 810 to detect the physiological characteristics of the specific user for two times to generate two PPG waveform signals when the change of blood pressure of specific user is at the linear state and non-linear state respectively.

If the reference measurement result received by the receiving unit 805 is measured and obtained when the change of blood pressure of specific user is at the linear state, the physiological sensor 810 is activated to detect the physiological characteristics of the specific user for one time to generate one PPG waveform signal when the change of blood pressure of specific user is at the same linear state, so that the processing circuit 815 can adjust corresponding parameter factor(s) of the basic/general blood pressure estimation model to generate the personal static blood pressure estimation model based on the reference measurement result and PPG waveform signal both corresponding to the linear state.

For an example of linear state (static blood pressure), the device 800 may assume or require that the specific user operates the reference sphygmomanometer to measure blood pressure in the morning after the user gets up for a few minutes or at the night before the user goes to bed, and the processing circuit 815 can control the physiological sensor 810 to detect the physiological characteristics of the specific user for one time to generate one PPG waveform signal after the user gets up for a few minutes or before the user goes to bed. In this example, it is not required for the device 800 to ask or command the specific user to operate the reference sphygmomanometer; the device 800 receives the reference measurement result of the reference sphygmomanometer and uses the physiological sensor 810 to perform physiological characteristics detection merely after the user gets up for a few minutes or before the user goes to bed. In other examples, the device 800 can be designed to ask or command the specific user to operate the reference sphygmomanometer. This is not intended to be a limitation.

Alternatively, if the reference measurement result received by the receiving unit 805 is measured and obtained when the change of blood pressure of specific user is at the non-linear state, the physiological sensor 810 is activated to detect the physiological characteristics of the specific user for one time to generate one PPG waveform signal when the change of blood pressure of specific user is at the same non-linear state, so that the processing circuit 815 can adjust corresponding parameter factor(s) of the basic/general blood pressure estimation model to generate the personal dynamic blood pressure estimation model based on the reference measurement result and PPG waveform signal both corresponding to the non-linear state.

For an example of non-linear state (dynamic blood pressure), the device 800 may assume or require that the specific user operates the reference sphygmomanometer to measure dynamic blood pressure after exercise to know his/her dynamic blood pressure when he/she is exercising, and the processing circuit 815 can control the PPG sensor 810 to detect the physiological characteristics of the specific user for one time to generate one PPG waveform signal after the user exercises. In this example, it is not required for the device 800 to ask or command the specific user to operate the reference sphygmomanometer; the device 800 receives the reference measurement result of the reference sphygmomanometer and uses the physiological sensor 810 to perform physiological characteristics detection merely after the user exercise. In other examples, the device 800 can be designed to ask or command the specific user to operate the reference sphygmomanometer. This is not intended to be a limitation.

In practice, the electronic device 800 may be designed to have multiple different default settings such as a morning mode setting, a night mode setting, and/or an exercise mode setting (e.g. jogging, swimming, or others), and can be implemented using an interactive human-machine interface device which can receive/accept input of the specific user and send a signal to ask/command the specific user to perform an action or behavior.

For instance, the specific user may select the morning mode setting or night mode setting, and the electronic device 800 is arranged to send a signal to ask or command the specific user to measure the static blood pressure by using the reference sphygmomanometer when the specific user gets up for a few minutes or before the specific user goes to bed; in other times, the device 800 does not send a signal to ask or command the specific user to measure the static blood pressure. The processing circuit 815 of electronic device 800 can be arranged to send a signal to instruct the specific user to input the values of measured static blood pressure which can be received by the receiving unit 805. The processing circuit 815 can be arranged to control the physiological sensor 810 to detect the physiological characteristics of the specific user to generate PPG waveform signal(s) at the same time or before/after the specific user inputs the values of measured static blood pressure. Thus, the processing circuit 815 can obtain/calculate the estimation result based on the PPG waveform signal(s) wherein the obtained reference measurement result and the estimation result both correspond to an identical/similar user behavior or action. In other words, the obtained reference measurement result and the calculated estimation result both are associated with the linear state for the blood pressure of the specific user.

Also, the specific user may select the exercise mode setting, and the device 800 is arranged to send a signal to ask or command the specific user to measure the dynamic blood pressure by using the reference sphygmomanometer after/when the specific user does an exercise; the device 800 does not send a signal to ask or command the specific user to measure the dynamic blood pressure by using the reference sphygmomanometer if detecting that the specific user is not exercising. The processing circuit 815 of device 800 can be arranged to send a signal to instruct the specific user to input the values of measured dynamic blood pressure which can be received by the receiving unit 805. The processing circuit 815 can be arranged to control the physiological sensor 810 to detect the physiological characteristics of the specific user to generate PPG waveform signal(s) when/after the specific user inputs the values of measured dynamic blood pressure. Thus, the processing circuit 815 can obtain/calculate the estimation result based on the PPG waveform signal(s) wherein the obtained reference measurement result and the estimation result both correspond to an identical/similar user behavior or action. In other words, the obtained reference measurement result and the calculated estimation result both are associated with the non-linear state for the blood pressure of the specific user.

In other embodiments, the processing circuit 815 can be also arranged to control the physiological sensor 810 to detect the physiological characteristics of the specific user to generate PPG waveform signal(s) by the specific user's control or when the specific user determines to use the estimation of physiological sensor 810. For example, the specific user may select an indoor mode setting of device 800 which corresponds to the linear state of blood pressure estimation, and the specific user may determine when and whether to activate/trigger the physiological sensor 810 to generate PPG waveform signal(s) if the specific user considers some timing is appropriate to estimate the static blood pressure. Similarly, the specific user may select the exercise mode setting corresponding to the non-linear state of blood pressure estimation, and the specific user may determine when and whether to activate/trigger the physiological sensor 810 to generate PPG waveform signal(s) if the specific user considers some timing is appropriate to estimate the dynamic blood pressure.

Further, the device 800 may be designed to be externally and electrically connected to the reference sphygmomanometer via wired/wireless communication, and the receiving unit 805 may be configured to directly receive the reference measurement result from the reference sphygmomanometer without instructing the user to input such measurement result. That is, the device 800 may immediately receive the reference measurement result from the reference sphygmomanometer after the user operates the sphygmomanometer. Then, the device 800 may automatically activate or trigger the physiological sensor 810 to detect the physiological characteristics of the specific user to generate PPG waveform signal(s). In other words, the device 800 can be designed to merely ask/instruct the specific user to use the reference sphygmomanometer, and then automatically receive the reference measurement result and trigger the physiological sensor 810 to generate PPG waveform signal(s).

In addition, for automatically generating a PPG waveform signal for static blood pressure estimation, the device 800 may incorporate with a sleep monitor function which can be used to detect when the user falls asleep and when the user gets up. When detecting the specific user falls asleep or gets up, the processor 815 controls the physiological sensor 810 to automatically detect the physiological characteristics of the specific user to generate a PPG waveform signal.

In Step 720A and Step 720B, for generating personal static and dynamic blood pressure models, the processing circuit 815 is arranged to calculate and obtain an estimation result of the static blood pressure of the specific user according to a generated PPG waveform signal at linear state and to obtain an estimation result of the dynamic blood pressure of the specific user according to a generated PPG waveform signal at non-linear state. In practice, for generating an estimation result, the processing circuit 815 may calculate and obtain the estimation result of blood pressure (either static or dynamic) of the specific user based on an interval between a major peak and a second peak of one PPG waveform signal. The second peak means a reflective wave. However, this is not meant to be a limitation. The processing circuit may perform calculation based on other algorithms and the PPG waveform signal to generate an estimation result.

In Step 725A and Step 725B, the processing circuit 815 is arranged to adjust two sets of parameter factor(s) of the basic/general blood pressure estimation model used by the physiological sensor 810 by comparing the at least two reference measurement results with at least two estimation results of blood pressure respectively, to generate the personal static and dynamic blood pressure estimation models dedicated for the specific user. For example, each reference measurement result has a reference value of systolic blood pressure and a reference value of diastolic blood pressure, and each estimation result of blood pressure has an estimation value of systolic blood pressure and an estimation value of diastolic blood pressure. For instance, the device 800 is arranged to estimate and obtain the estimation value of systolic blood pressure and estimation value of diastolic blood pressure based on waveform component intervals of the generated PPG waveform signal.

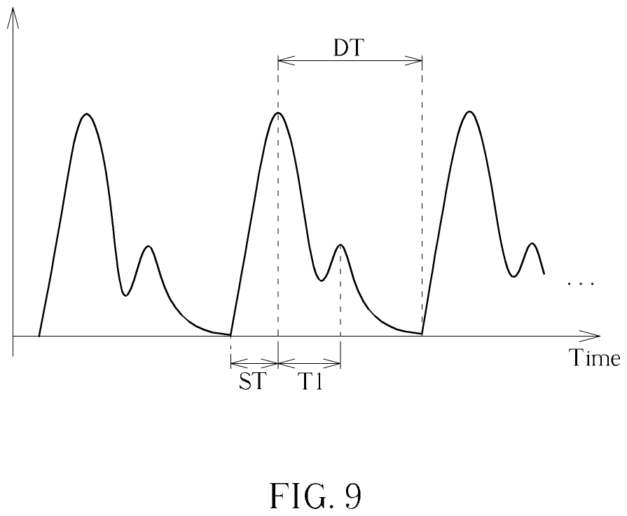

Refer to FIG. 9, which is a diagram showing an example of one PPG waveform signal. As shown in FIG. 9, each repeated waveform component of the PPG waveform signal has three different time intervals ST, DT, and T1. ST means the time interval between the start of the repeated waveform component and the maximum value (i.e. the major peak) of the repeated waveform component. DT means the time interval between the end of the repeated waveform component and the maximum value of the repeated waveform component. T1 means the time interval between the maximum value of the repeated waveform component and the second maximum value (i.e. second peak) of the repeated waveform component.

The basic/general blood pressure estimation model can be represented by the following equations:

ESBP=A1×DT+A2

EDBP=B1×T1+B2

wherein A1, B1, A2, and B2 are basic/general parameter factors, and ESBP and EDBP respectively indicate the estimation values of systolic blood pressure and diastolic blood pressure to be calculated. For example, A1 is equal to −0.095, and B1 is equal to −0.344. A2 is equal to 188.581, and B2 is equal to 174.308. However, this is not intended to be a limitation. The basic/general parameter factors may be configured as different values in other examples.

The processing circuit 815 is arranged to compare the reference value of systolic blood pressure with the estimation value of systolic blood pressure (i.e. ESBP) to calculate a difference which is used as a reference (a regulating/calibration parameter) to regulate or adjust the parameter factor(s) A1 and/or A2. Also, the processing circuit 815 is arranged to compare the reference value of diastolic blood pressure with the estimation value of diastolic blood pressure (i.e. EDBP) to calculate a difference which is used as a reference (another regulating/calibration parameter) to regulate or adjust the parameter factor(s) B1 and/or B2. By respectively perform comparison for static blood pressure and dynamic blood pressure, the device 800 can calculate and obtain the personal blood pressure estimation model (personal static and dynamic blood pressure estimation models) dedicated for a specific user. Based on the personal blood pressure estimation model of device 800, the specific user or person can use the device 800 to automatically estimate and derive more accurate values of his/her blood pressure no matter what behavior or action the user or person is doing now.

Further, the device 800 may be arranged to generate a personal blood pressure estimation model having a set of parameter factors only for systolic blood pressure or only for diastolic blood pressure in response to a particular user's requirement or control. For example, the particular user may measure the reference value of only systolic blood pressure or only diastolic blood pressure by using the reference sphygmomanometer, and the device 800 can be arranged to calculate and adjust the parameter factors only for systolic blood pressure or only for diastolic blood pressure to form the personal blood pressure estimation model dedicated to the particular user.

Additionally, in other embodiments, the device 800 can more precisely define or identify the linear and non-linear states for the change of blood pressure of the specific user according to the heart rate of the specific user, so as to generate and obtain more accurate personal blood pressure estimation model. The device 800 is capable of precisely detecting or measuring a current value, a minimum value, and a maximum value of the specific user's heart rate around all day. For example, the device 800 can be implemented as a wearable electronic device which can be arranged to automatically detecting the specific user's heart rate so as to obtain the minimum value and maximum value of heart rate. The minimum value of heart rate can be defined as the value of specific user's rest heart rate corresponding to the steady state of specific user's blood pressure. So, the device 800 can derive a heart rate reserve percentage P according to the following equation:

$$P = \frac{( E - R )}{( MAX - R )} \times 100 \%$$

wherein parameter R indicates the value of rest heart rate of the specific user, parameter E indicates a currently measured value of heart rate of the specific user, parameter MAX indicates the maximum value of heart rate of the specific user. It should be noted that the maximum value of heart rate of the specific user can be inputted or modified by the specific user in other embodiments.

A steady state for the change of blood pressure means that the heart rate reserve percentage currently measured is equal to zero. For example, the specific user at the steady state may mean that the specific user takes a rest for a few minutes. The device 800 is to define/configure a range of heart rate reserve percentage from zero to a first percentage value such as 10% as the linear state of blood pressure for the specific user, and to define/configure another range of heart rate reserve percentage from the first percentage value such as 10% to a second percentage value such as 100% as the non-linear state of blood pressure for the specific user. For example, the specific user is walking around a room (but not limited), and the heart rate reserve percentage currently measured may be at the linear state. The specific user is jogging outdoors, and the heart rate reserve percentage currently measured may be at the non-linear state.

The electronic device 800 is arranged to estimate the current value of the specific user's heart rate by using the physiological sensor 810, and then the processing circuit 815 is arranged to calculate or derive the value of heart rate reserve percentage currently measured. Thus, the processing circuit 815 can accurately determine that the change of specific user's blood pressure is at the linear state or non-linear state. If the processing circuit 815 determines that the change of specific user's blood pressure is at the linear state, the processing circuit 815 is arranged to compare the reference value of static blood pressure measured by the reference sphygmomanometer with the estimation value of static blood pressure calculated based on information of physiological sensor 810, to adjust a set of corresponding parameter factor(s) of the basic/general blood pressure estimation model so as to finally generate the personal static blood pressure estimation model.

Similarly, if the processing circuit 815 determines that the change of specific user's blood pressure is at the non-linear state, the processing circuit 815 is arranged to compare the reference value of dynamic blood pressure measured by the reference sphygmomanometer with the estimation value of dynamic blood pressure calculated based on information of physiological sensor 810, to adjust another set of corresponding parameter factor(s) of the basic/general blood pressure estimation model so as to finally generate the personal dynamic blood pressure estimation model.

Based on the information of heart rate reserve percentage of the specific user, the device 800 can precisely distinguish the linear state from the non-linear state for the change of specific user's blood pressure no matter what actions/behavior the specific user does. Thus, a more accurate personal blood pressure estimation model having static and dynamic estimation models can be obtained.

Further, it should be noted that the electronic device 800 can be also arranged to generate and obtain merely the personal static blood pressure estimation for the specific user or generate and obtain merely the personal dynamic blood pressure estimation for the specific user. FIG. 7 shows a preferred embodiment. This is not intended to be a limitation.

To summarize, the invention aims at providing a solution of accurately obtaining an estimation result of the physiological feature of a particular user based on only one observation and his/her unique profile information associated with the physiological feature. In addition, the invention also aims at providing a solution of establishing different unique profiles for the physiological feature of different users and a solution of managing and evaluating different profiles of the physiological feature of different users for big data analysis. In addition, the invention also aims at providing a method which is capable of establishing or generating a personal blood pressure estimation model for a specific user/person merely based on a basic or common blood pressure estimation model so as to more accurately estimate the specific user/person's blood pressure even though the specific user/person may operate/press/touch the electronic device with different pressures.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method to be executed on a mobile device, a handheld device, or a wearable electronic device and arranged for establishing a personal blood pressure estimation model dedicated for a specific user operating the mobile device, the handheld device, or the wearable electronic device, comprising:
receiving a first reference measurement result generated by a reference sphygmomanometer which is a larger external electronic device being not like the mobile device, the handheld device, and the wearable electronic device;
using a physiological sensor, installed on the mobile device, the handheld device, or the wearable electronic device, to measure the specific user's blood pressure to generate a first photoplethysmogram signal to calculate a first estimation result of the specific user's blood pressure, comprising:
using the physiological sensor under a normal sensing mode to sense the specific user's blood pressure to generate the first photoplethysmogram signal of the normal sensing mode;
determining a matched photoplethysmogram signal from a plurality of test photoplethysmogram signals by comparing the plurality of test photoplethysmogram signals with the first photoplethysmogram signal of the normal sensing mode;
when the matched photoplethysmogram signal is an optimal test photoplethysmogram, calculating the first estimation result of the specific user's blood pressure according to the matched photoplethysmogram signal; and
when the matched photoplethysmogram signal is a non-optimal test photoplethysmogram signal in the plurality of test photoplethysmogram signals and different from the optimal test photoplethysmogram signal, calculating a preliminary estimation of the specific user's blood pressure according to the non-optimal test photoplethysmogram signal and then compensating the preliminary estimation by referring to a compensation parameter corresponding to the non-optimal test photoplethysmogram signal without using the optimal test photoplethysmogram signal so as to generate the first estimation result of the specific user's blood pressure, wherein a plurality of non-optimal test physiological signals in the plurality of test photoplethysmogram signals are respectively generated in response to different pressures asserted by the specific user on the physiological sensor; and
generating a first regulating parameter by comparing the first reference measurement result with the first estimation result to calculate a first difference between the first reference measurement result and the first estimation result as the first regulating parameter;
establishing a set of parameter factor(s) of the personal blood pressure estimation model dedicated for the specific user operating the mobile device, the handheld device, or the wearable electronic device by using the first regulating parameter to adjust a set of parameter factor(s) of a basic blood pressure estimation model implemented on the mobile device wherein the set of parameter factor(s) of the basic blood pressure estimation model are used and identical for the specific user and another different user while the set of parameter factor(s) of the personal blood pressure estimation model is merely used for the specific user and is not used for the another different user; and
using the physiological sensor with the set of parameter factor(s) of the established personal blood pressure estimation model dedicated for the specific user to measure the specific user's blood pressure to generate blood pressure information without asking the specific user to operate the reference sphygmomanometer.

2. The method of claim 1, wherein the first reference measurement result and the first estimation result correspond to an identical state corresponding to a change of the specific user's blood pressure.

3. The method of claim 2, wherein the identical state is either one of a linear state or a non-linear state; the linear state indicates that a value of the specific user's blood pressure linearly changes with a change of the specific user's heart rate, and the non-linear state indicates that the value of the specific user's blood pressure non-linearly changes with the change of the specific user's heart rate.

4. The method of claim 2, further comprising:
detecting a heart rate reserve percentage of the specific user; and
configuring a range of the heart rate reserve percentage to be associated with the identical state;
wherein the first reference measurement result and the first estimation result are generated and obtained when the detected heart rate reserve percentage falls within the range.

5. The method of claim 1, further comprising:
sending a signal to instruct the specific user to operate the reference sphygmomanometer before receiving the first reference measurement result of the reference sphygmomanometer.

6. The method of claim 1, further comprising:
receiving a second reference measurement result of the reference sphygmomanometer;
using the physiological sensor to measure the specific user's blood pressure to generate a second photoplethysmogram signal;
calculating a second estimation result of the specific user's blood pressure according to the second photoplethysmogram signal;
generating a second regulating parameter by comparing the second reference measurement result with the second estimation result; and
establishing the personal blood pressure estimation model by further using the second regulating parameter to adjust another set of parameter factor(s) of the basic blood pressure estimation model.

7. The method of claim 6, wherein the first reference measurement result and the first estimation result correspond to a first state corresponding to a change of the specific user's blood pressure, and the second reference measurement result and the second estimation result correspond to a second state corresponding to the change of the specific user's blood pressure.

8. The method of claim 7, wherein the first state is a linear state which indicates that a value of the specific user's blood pressure linearly changes with a change of the specific user's heart rate, and the second state is a non-linear state which indicates that the value of the specific user's blood pressure non-linearly changes with the change of the specific user's heart rate.

9. The method of claim 8, wherein the personal blood pressure estimation model comprises a personal static blood pressure estimation model and a personal dynamic blood pressure estimation model.

10. The method of claim 8, further comprising:
detecting a heart rate reserve percentage of the specific user; and
configuring a first range of the heart rate reserve percentage to be associated with the linear state and a second range of the heart rate reserve percentage to be associated with the non-linear state;
wherein the first reference measurement result and the first estimation result are generated and obtained when the detected heart rate reserve percentage falls within the first range, and the first reference measurement result and the first estimation result are generated and obtained when the detected heart rate reserve percentage falls within the second range.

11. An electronic device being a mobile device, a handheld device, or a wearable electronic device and arranged for establishing a personal blood pressure estimation model dedicated for a specific user operating the mobile device, the handheld device, or the wearable electronic device, comprising:
a receiving unit, configured to receive a first reference measurement result of a reference sphygmomanometer which is a larger external electronic device being not like the mobile device, the handheld device, and the wearable electronic device;
a physiological sensor, coupled to the receiving unit and installed on the mobile device, the handheld device, or the wearable electronic device, configured to measure the specific user's blood pressure to generate a first photoplethysmogram signal; and
a processing circuit, coupled to the receiving unit and the physiological sensor, configured to:
calculate a first estimation result of the first blood pressure of the specific user according to the first photoplethysmogram signal;
generate a first regulating parameter by comparing the first reference measurement result with the first estimation result to calculate a first difference between the first reference measurement result and the first estimation result as the first regulating parameter;
establish a set of parameter factor(s) of the personal blood pressure estimation model dedicated for the specific user operating the mobile device, the handheld device, or the wearable electronic device by using the first regulating parameter to adjust a set of parameter factor(s) of a basic blood pressure estimation model implemented on the mobile device wherein the set of parameter factor(s) of the basic blood pressure estimation model are used and identical for the specific user and another different user while the set of parameter factor(s) of the personal blood pressure estimation model is merely used for the specific user and is not used for the another different user; and
use the physiological sensor device with the established personal blood pressure estimation model dedicated for the specific user to measure the specific user's blood pressure to generate blood pressure information without asking the specific user to operate the reference sphygmomanometer;
wherein the physiological sensor is arranged for sensing the specific user's blood pressure under a normal sensing mode to generate the first photoplethysmogram signal of the normal sensing mode; and, the processing circuit is arranged for determining a matched photoplethysmogram signal from a plurality of test photoplethysmogram signals by comparing the plurality of test photoplethysmogram signals with the first photoplethysmogram signal of the normal sensing mode; when the matched photoplethysmogram signal is an optimal test photoplethysmogram signal, the processing circuit calculates the first estimation result of the specific user's blood pressure according to the matched photoplethysmogram signal; and, when the matched photoplethysmogram signal is a non-optimal test photoplethysmogram signal in the plurality of test photoplethysmogram signals and different from the optimal test photoplethysmogram signal, the processing circuit calculates a preliminary estimation of the specific user's blood pressure according to the non-optimal test photoplethysmogram signal and then compensates the preliminary estimation by referring to a compensation parameter corresponding to the non-optimal test photoplethysmogram signal without using the optimal test photoplethysmogram signal so as to generate the first estimation result of the specific user's blood pressure, wherein a plurality of non-optimal test physiological signals in the plurality of test photoplethysmogram signals are respectively generated in response to different pressures asserted by the specific user on the physiological sensor.

12. The electronic device of claim 11, wherein the first reference measurement result and the first estimation result correspond to an identical state corresponding to a change of the specific user's blood pressure.

13. The electronic device of claim 12, wherein the identical state is either one of a linear state or a non-linear state; the linear state indicates that a value of the specific user's blood pressure linearly changes with a change of the specific user's heart rate, and the non-linear state indicates that the value of the specific user's blood pressure non-linearly changes with the change of the specific user's heart rate.

14. The electronic device of claim 12, wherein the processing circuit controls the physiological sensor to detect a heart rate reserve percentage of the specific user; the processing circuit configures a range of the heart rate reserve percentage to be associated with the identical state; and, the first reference measurement result and the first estimation result are generated and obtained when the processing circuit decides that the detected heart rate reserve percentage falls within the range.

15. The electronic device of claim 11 is an interactive human-machine interface device which is configured to send a signal to instruct the specific user to operate the reference sphygmomanometer before receiving the first reference measurement result of the reference sphygmomanometer.

16. The electronic device of claim 11, wherein the receiving unit is arranged to receive a second reference measurement result of the reference sphygmomanometer; the physiological sensor is used to measure the specific user's blood pressure to generate a second photoplethysmogram signal; and, the processing circuit is arranged for: calculating a second estimation result of the specific user's blood pressure according to the second photoplethysmogram signal; generating a second regulating parameter by comparing the second reference measurement result with the second estimation result; and, establishing the personal blood pressure estimation model by further using the second regulating parameter to adjust another set of parameter factor(s) of the basic blood pressure estimation model.

17. The electronic device of claim 16, wherein the first reference measurement result and the first estimation result correspond to a first state corresponding to a change of the specific user's blood pressure, and the second reference measurement result and the second estimation result correspond to a second state corresponding to the change of the specific user's blood pressure.

18. The electronic device of claim 17, wherein the first state is a linear state which indicates that a value of the specific user's blood pressure linearly changes with a change of the specific user's heart rate, and the second state is a non-linear state which indicates that the value of the specific user's blood pressure non-linearly changes with the change of the specific user's heart rate.

19. The electronic device of claim 18, wherein the personal blood pressure estimation model comprises a personal static blood pressure estimation model and a personal dynamic blood pressure estimation model.

20. The electronic device of claim 18, wherein the processing circuit is arranged to control the physiological sensor to detect a heart rate reserve percentage of the specific user; and the processing circuit configures a first range of the heart rate reserve percentage to be associated with the linear state and a second range of the heart rate reserve percentage to be associated with the non-linear state; the first reference measurement result and the first estimation result are generated and obtained when the processing circuit decides that the detected heart rate reserve percentage falls within the first range, and the first reference measurement result and the first estimation result are generated and obtained when the processing circuit decides that the detected heart rate reserve percentage falls within the second range.
